(19) United States
(12) Patent Application Publication
(10) Pub. No.: US 2024/0200058 A1
(43) Pub. Date: Jun. 20, 2024

(54) METHODS AND APPRATUS FOR PROTEIN AND PEPTIDE SEQUENCING

(71) Applicant: Shifeng LI, Freemont, CA (US)

(72) Inventor: Shifeng LI, Freemont, CA (US)

(21) Appl. No.: 18/529,329

(22) Filed: Dec. 5, 2023

Related U.S. Application Data
(60) Provisional application No. 63/430,347, filed on Dec. 6, 2022.
(60) Provisional application No. 63/436,869, filed on Jan. 3, 2023.
(60) Provisional application No. 63/436,881, filed on Jan. 4, 2023.
(60) Provisional application No. 63/437,324, filed on Jan. 5, 2023.

(51) Int. Cl. C12N 15/10; G01N 33/53; G01N 33/68

(52) U.S. Cl. CPC C12N 15/1065; G01N 33/5308; G01N 33/6824

(57) ABSTRACT

This disclosure is directed to a method for sequencing protein and peptide. The method specifies the steps of providing a polypeptide that is immobilized on solid surfaces and binding a N-terminal amino acid (NTAA) of the polypeptide with a modification molecule. Further, the method requires contacting the polypeptide with a NTAA binding molecule with a DNA coding tag, transferring an information from the DNA coding tag of the NTAA binding molecule to a universal primer and then forming an extended DNA tag on the solid surfaces, and cleaving the N-terminal amino acid on the polypeptide. Then, step (b) through step (e) are cyclically repeated. Finally, extended DNA strains are decoding on the solid surfaces.

### CROSS-REFERENCE TO RELATED APPLICATIONS

The present application claims priority to U.S. Provisional Patent Application No. 63/430,347, filed on Dec. 6, 2022, titled “Methods and Apparatus for Protein and Peptide Sequencing”; U.S. Provisional Patent Application No.63/436,869, filed on Jan. 3, 2023, titled “Methods and Apparatus for Protein and Peptide Sequencing”; U.S. Provisional Patent Application No. 63/436,881, filed on Jan. 4, 2023, titled “Methods and Apparatus for Protein and Peptide Sequencing”; U.S. Provisional Patent Application No. 63/437,324, filed on Jan. 5, 2023, titled “Methods and Apparatus for Protein and Peptide Sequencing”; the disclosures of which applications are incorporated by reference in its entirety for all purposes.

### FIELD OF THE INVENTION

The present disclosure relates to methods and apparatus for sequencing of proteins and polypeptides. In some embodiments, the methods can sequence a pool of proteins and polypeptides from multiple samples.

### BACKGROUND OF THE INVENTION

Proteins are large biomolecules and macromolecules that comprise one or more long chains of amino acidresidues. Proteins perform a vast array of functions with organisms, including catalysing metabolic reactions, DNA replication, responding to stimuli, providing structure to cells and organisms, and transporting molecules from one location to another. Protein differs from one another primarily in their sequence of amino acids, which is dictated by the nucleotide sequence of their genes, and which usually results in protein folding into a specific 3D structure that determines its activity. A linear chain of amino acid residues is called a polypeptide. A protein contains at least one long polypeptide. The individual amino acid residues are bonded together by peptide bonds and adjacent amino acid residues. The sequence of amino acid residues in a protein is defined by the sequence of a gene, which is encoded in the genetic code. Shortly after synthesis, the residues in a protein are often chemically modified by post translation modification (PTM), which alters the physical and chemical properties, folding, stability, activity, and ultimately, the function of the proteins. The repertoire of different protein molecules is extensive, thus proteins contain a vast amount of information that is largely unexplored. Yet the protein information is directly needed for a better understanding of proteome dynamics in health and disease and to help enable precision medicine. As such, there is a great need to develop high throughput tools to collect the vast amount of proteomic information.

Within the last two decades, next generation of sequencing (NGS) has achieved tremendous progress with throughout, scalability and speed (Levene et al., 2003, Science, 299, 682-686; Drmanac et al, 2010, Science, 327, 78-81; Rothberg et al, Nature, 2011, 475, 348-352; Zhao et al., PLOS, 2017, 1-9; Arslan et al., Nature Biotech., 2023). The sequencing throughput increased from initial megabytes (Mb) to terabytes (Tb), the speed of the sequencing reduced from initial weeks to today hours and the cost of whole genome sequencing (WGS) decreased from an estimated above million dollars and today it is approximately 100 dollars. In the field of genomics, NGS has transformed the field by enabling analysis of billions of DNA sequences in a single instrument run, whereas the development of next generation high throughput protein and peptide sequencing is still in the early stage.

Peptide sequencing based on Edman degradation was first developed by Pehr Edman in 1950. A stepwise degradation of the N-terminal amino acid on a peptide through a series of chemical modification and downstream HPLC or mass spectrometry analysis (Laursen et al., 1971, Eur. J. Biochem. 20,89-102; Niall et al., 1973, Sequence Determination, 36, 942-1010; Smith et al., 2001, Encyclopedia of Life Sciences, 1-3). Phenyl isothiocyanate is reacted with an uncharged N-terminal amino group, under mildly alkaline conditions, to form a cyclical phenylthiocarbamoyl derivative. Then, under acidic conditions, the derivative of the terminal amino acid is cleaved as a thiazolinone derivative. The thiazolinone amino acid is then selectively extracted into an organic solvent and treated with acid to form the more stable phenylthiohydantoin (PTH)—amino acid derivative that can be identified by using chromatography or electrophoresis. Then, the procedure is repeated in a stepwise to identify all amino acids along a peptide. The major drawback of this technique is that the sequencing length cannot be more than 50 to 60 residues, in practice under 30, because the cyclic derivation is not always going to completion.

In the last 10 to 15 years, peptide sequencing using MALDI, electrospray mass spectroscopy (MS) and LC-MS/MS has largely replaced Edman degradation protein sequencing. Despite the recent advances in MS instrumentation, MS still suffers from several drawbacks including high instrument cost, requirement for a sophisticated user, poor quantification ability, and limited dynamic range of the proteome. For example, since proteins ionize at different levels of efficiencies, absolute quantitation and even relative quantitation between samples is still challenging. MS typically only analyzes more abundant species, making characterization of low abundance protein challenging. Finally, sequencing throughout is typically limited to a few thousand peptides per run, which is inadequate for bottom-up high throughput proteome analysis. Furthermore, there is a significant compute requirement to deconvolute thousands of complex MS spectra recorded for each sample.

In last a few years, next generation protein sequencing (NGPS) technology became more and more popular. Similar with NGS, NGPS also utilizes highly parallelized format to achieve high throughput for a run. However, unlike NGS, NGPS cannot amplify peptide copy numbers to boost sequencing signal, so it is a single molecule level sequencing technology. Many methods are implemented to boost protein sequencing signal. For examples, Erysion utilizes total internal reflection fluorescence microscopy (TIRFM) and Edman degradation to sequence protein. TIRFM can reduce the background so the single molecule fluorescence signal can be reliably detected in a cyclic way (Swaminathan et al., PLOS, 2015, 1-17; Swaminathan et al., Nature Biotech., 2018, 36, 1076-1082). Quantum Si uses zero mode waveguide to amplify fluorescence signal from dye molecules conjugated on N-terminal amino acid (NTAA) binding molecules. The dynamic information of the fluorescence, like lifetime and pulse duration etc., can be utilized to differentiate most of amino acids along a peptide (Reed et al., Science, 2022, 378, 186-192). Encodia implements a novel method. First, DNA extension or ligation cyclically converts amino acids on a polypeptide into DNA barcodes along single or multiple DNA strains and then DNA sequencing technology, like single molecule DNA sequencing or next generation DNA sequencing, decodes these DNA barcodes, which subsequently decode the sequence of proteins or peptides (Chee et al., 2022, U.S. Pat. No. 11,513,126B2; Chee et al., 2022, US2022/0214353A1; Chee et al., 2021, 2021/0405058A1; Chee et al., 2021, 2021/0396762A1; Beierle et al., 2020, US2020/0348307A1)

Accordingly, there is a need for highly parallelized, accurate, sensitive protein sequencing technology that can accommodate the entire sequencing workflow within single instrument. The present disclosure fulfills these needs.

These and other aspects of the invention will be apparent upon reference to the following detailed description. To this end, various references are set forth herein which described in more detail certain background information, procedure, compounds and/or compositions, and are each hereby incorporated by reference in their entirety.

### SUMMARY OF THE INVENTION

The summary is not intended to be used to limit the scope of the claimed subject matter. Other features, details, utilities, and advantages of the claimed subject matter will be apparent from the detailed description including those aspects disclosed in the accompanying drawings and in the appended claims.

Provided in some aspects are methods for sequencing a protein or a polypeptide, comprising the steps of: (a) providing the polypeptide immobilized on solid surfaces optionally through a sample ID DNA tag; (b) optionally binding the N-terminal amino acid (NTAA) of the polypeptide with a modification molecule; (c) contacting the polypeptide with a NTAA binding molecule with a coding DNA tag; (d) transferring the information of the coding DNA tag to a primer on solid surfaces; (e) cleaving the N-terminal amino acid on the polypeptide; (f) cyclically repeating step (b) through step (e); (g) amplifying the coding DNA strands into a cluster In situ on solid surfaces; (h) decoding DNA strands on the solid surfaces. In some embodiments, the polypeptide is directly immobilized on the solid surfaces without a coding DNA tag.

In some embodiments, the chemical reagent in step (b) is a modification molecule that can chemically binds with a N-terminal amino acid, thus the presence of this modification molecule can increase the binding specificity of the NTAA binding molecules.

In some embodiments, the decoding method in step (f) is nucleic acid hybridization assay or nucleic acid sequencing.

Provided in other aspects is a solid support for protein and peptide sequencing. The solid support can be beads, silicon, glass, sapphire, or metal substrates to immobilize polypeptides. The polypeptides are immobilized in either random or regular array format on the solid support.

### BRIEF DESCRIPTION OF THE DRAWINGS

Non-limiting embodiments of the present invention will be described by way of example with reference to the accompanying figures, which are schematics are not intended to be drawn to scale. For purposes of illustration, not every component is labeled in every figure, nor is every component of each embodiment of the invention shown where illustration is not necessary to allow those of ordinary skill in the art to understand the invention.

FIG. 1 illustrates a sequencing scheme for proteins and peptides. Thus, in one embodiment, provided herein is universal primers, a polypeptide with a sample ID DNA tag, a NTAA binding molecule with a barcode DNA tag, a modification molecule bound with the NTAA on the polypeptide and a reaction enzyme. The sample ID DNA tag on the polypeptide comprises one or more barcode sequences, for example sample ID or UMI. The barcode DNA tag on the NTAA comprises one or more barcode sequences, for example sample ID or UMI or specific sequence for cycle number, specific sequence for amino acid etc. In some embodiment, the components comprise universal primers, a polypeptide with a sample ID DNA tag, a NTAA binding molecule with a barcode DNA tag and a reaction enzyme. In some embodiment, the components comprise universal primers, a polypeptide, a NTAA binding molecule with a barcode DNA tag, and a reaction enzyme. In some embodiment, the components comprise universal primers, a polypeptide, a NTAA binding molecule with a barcode DNA tag, a modification molecule bound with the NTAA on the polypeptide and a reaction enzyme.

FIG. 2A to FIG. 2C illustrate the steps for protein and peptide sequencing in one embodiment. A polypeptide is directly immobilized on solid surface through a sample ID DNA tag. A modification molecule binds with a NTAA along the polypeptide and then the NTAA binding molecule with a barcode DNA tag is specific to identify the modified NTAA on the polypeptide. The barcode DNA tag is hybridized with a universal primer on the solid surfaces. Enzyme extends the primer and then transfer the barcode information to the universal primer on the solid surfaces. The modified NTAA is cleaved from the polypeptide using a chemical method or an enzymatic method and then expose next the NTAA along the polypeptide strand. The previous steps repeat cyclically to transfer all the barcodes to the solid surfaces. The DNA strands with barcode information are amplified into clusters in situ on the solid surfaces. These amplified DNA clusters are cyclically decoded using hybridization assay or sequencing.

FIG. 3 illustrates a general overview of a sample ID DNA tag of a polypeptide.

FIG. 4 illustrates a general overview of a coding DNA tag of a binding molecule.

FIG. 5A illustrates in situ DNA strand amplification in one embodiment. Sample ID DNA tags and extended DNA coding tags derived from single polypeptide are colocalized on the solid surfaces. Circular amplification primer templates are hybridized with these DNA tags and then amplification polymerases (e.g. Phi29) follow the circular templates to incorporate nucleotides into the DNA strands. After many cycles linear duplication, the DNA tags are amplified into clusters on the solid surfaces.

FIG. 5B illustrates in situ DNA strand amplification in another embodiment. Sample ID DNA tags and extended DNA coding tags derived from single polypeptide are colocalized on the solid surfaces. Nicked templates are captured by the surface immobilized primers. Surface primers are extended to the full length of the template by strand displacement enzyme at 37° C. Template invasion by solution phase primers and template walk to nearby surface primer to form two copies of the template.

FIG. 6A illustrates a method of decoding DNA strands using hybridization assay. A dye labelled ID barcode probe is to decode sample information in the cluster and a series of dye labelled barcode probes is to decode the information of the DNA coding tags in the same cluster on solid surfaces. The ID barcode probes are specific for every sample and the barcode probes are specific sequence of nucleic acids for each cycle. Using these ID barcodes probes and a batch of the barcode probes, the information of samples and the sequence of polypeptides can be sequentially decoded using hybridization assays.

FIG. 6B illustrates another method of decoding DNA strands using sequencing. Both the sample ID DNA tags and the extended DNA coding tags on solid surfaces have sequencing priming sites. In some embodiments, the sample ID DNA tags and the extended DNA coding tags share the same sequencing primer. After hybridizing this primer, the sample information of the sample ID DNA tags and the information of the extended DNA tags can be sequentially decoded in a stepwise method. In some embodiments, the sample ID DNA tag and the extended DNA coding tags in every cycle have unique sequencing primers. Thus, the sample information can only be decoded by a specific primer for the sample ID DNA tag and the information of extended DNA coding tags in Cycle N can only be decoded by a specific primer for Cycle N.

FIG. 7A is a representation of an exemplary sequencing system that accommodates a sequencing flow cell according to the present disclosure.

FIG. 7B is a representation of another exemplary sequencing system that accommodates a sequencing flow cell according to the present disclosure.

FIGS. 8A-8B illustrate two types of solid surfaces that can support protein and peptide sequencing: random array format and pattern array format.

FIG. 9A illustrates the overall layout of the structured arrays and detailing one arrangement of individual sites.

FIG. 9B illustrates the overall layout of the structured arrays and detailing another arrangement of individual sites.

### DETAILED DESCRIPTION

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a”, “an” and “the” include plural references unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

As used herein, the term “polypeptide” refers to a molecule comprising a chain of two or more amino acids joined by peptide bonds. In some embodiments, a polypeptide comprises 2 to 50 amino acids. In some embodiments, a polypeptide is a protein. In some embodiments, in addition to a primary structure, a protein comprises a secondary, territory, or higher structure. The amino acids of polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed, or be produced by a combination of methodologies as described above. Polypeptides may also comprise additional groups modifying the amino acid chain, for example, functional groups added via post-translational modification.

As used herein, the term “amino acid” refers to an organic compound comprising an amine group, a carboxylic acid group, and a side-chain specific to each amino acid, which serve as a monomeric subunit of a peptide. An amino acid includes 20 standard, naturally occurring or canonical amino acids as well as non-standard amino acids. The standard, naturally-occurring amino acids include Alanine (A or Ala), Cysterine (C or Cys), Aspartic Acid (D or Asp), Glutamic Acid (E or Glu), Phenylalanine (F or Phe), Glycine (G or Gly), Histidine (H or Glu), Isoleucine (I or lle), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp) and Tyrosine (Y or Tyr). An amino acid may be an L-amino acid or a D-amino acid. Non-standard amino acids may be modified amino acids, amino acid analogs, amino acid mimetics, non-standard proteinogenic amino acids or non-proteinogenic amino acids that occur naturally or are chemically synthesized.

As used herein, the term “post-translational modification” refers to modifications that occur on a peptide after its translation by ribosomes is complete. A post-translational modification may be a covalent modification or enzymatic modification. Examples of post-translation modifications include, but are not limited to Phosphorylation, N-glycosylation, O-glycosylation, Glyiation, C-glycosylation, Phosphoglycosylation, Ubiquitination, S-nitrosylation, Methylation, N-acetylation, Lipidation and Proteolysis etc.

As used herein, the term “modification molecule” refers to a nucleic acid molecule, a peptide, a polypeptide, a protein, carbonhydrate, or a small molecule that binds to, recognizes, or combines with a polypeptide or a component or subunit of a polypeptide. The modification molecule may form a covalent association or non-covalent association with the polypeptide or component or subunits of a polypeptide. A modification molecule may bind to an N-terminal peptide, a C-terminal peptide, or protein molecule. A modification molecule may exhibit selective binding to a component or subunit of a polypeptide. In some embodiments, a modification molecule may exhibit less selective binding, where the modification molecule is capable of binding to multiple of components or subunits of a polypeptide.

As used herein, the term “ligand” refers to any molecule or moiety with a functional group that are connected to the compounds to form a coordination complex. Ligand may refer to one or more ligands attached to a compound. In some embodiments, the ligand is a pendant group or binding sites.

As used herein, the term “proteome” can include the entire set of proteins, polypeptides, or peptides expressed by a genome, cell, tissue, or organism at a certain time. It is the set of expressed proteins in a given type of cell or organism, at a given time, under defined conditions. A cellular proteome is the collection of proteins found in a particular cell type under a particular set of environmental conditions. In some aspect, proteome refers to the collection of proteins in certain sub-cellular systems, such as organelles. As used herein, the term “proteomics” refers to quantitative analysis of the proteome within cells, tissues, and body fluids and the corresponding spatial distribution of the proteome within the cell and within tissues.

As used herein, the term “NTAA” refers to the terminal amino group with free amine functional group at the end of the peptide chain. The terminal amino acid at the other end of the chain that has a free carboxyl group is referred to herein as the “C-terminal amino acid” (CTAA).

As used herein, the term “barcode” refers to a unique sequence associated with a polynucleotide. This chain may have 2 to about 30 bases of nucleic acid units and the unique nucleic acid sequence provides an identification or origin information for an amino acid, a polypeptide, or reaction cycle, a set of samples etc. In certain embodiments, each barcode within a population of barcodes is different. Barcode can be computationally deconvoluted derived from an individual polypeptide, sample, library, etc. A barcode can also be used for deconvolution of collection of polypeptides that have been distributed into small compartments for enhanced mapping. For example, rather than mapping a peptide back to the proteome, the peptide is mapped back to its originating protein molecule or protein complex.

As used herein, the term “sample ID” refers to a barcode that identifies from which sample a polypeptide derives or come from.

As used herein, the term “coding tag” refers to a polynucleotide with unique

sequence identifying information for its associated chemical agent. A coding tag may also be comprised of an optional UMI and/or an optional reaction cycle-specific barcode. In certain embodiments, a coding tag may further comprise a reaction cycle specific barcode, a unique molecular identifier, a universal primming site, or any combination thereof.

As used herein, the term “universal primer” refers to a polynucleotide molecule, which may be used for library amplification, extension, ligation and/or for sequencing reactions. In some aspect, a universal primer can be used for amplification. For example, extended DNA tag molecules from a universal primer can be used for rolling circle amplification to form DNA nanoballs that can be used as sequencing templates. Alternatively, extended DNA tag molecules may be amplificated into clusters in situ and then sequenced by polymerase extension from universal primers.

As used herein, the term “solid support” or “solid surfaces” or “substrate” refers to any solid materials to which a polypeptide can be attached directly or indirectly by covalent or non-covalent interactions, or any combination thereof. A solid support may be two-dimensional planar surface or three-dimensional surface. A solid support can be any support surface including, but not limited to, a bead, a microbead, an array, a glass surface, a silicon surface, a plastic surface etc. Materials for a solid support include but are not limited to acrylamide, agarose, cellulose, glass, gold, quartz, polystryrene, polyethylene, plyethylene oxide, polysilicates, polycarbonates, Teflon, fluorocarbons, nylon, functionalized silane, collagen, polyamino acids, or any combination thereof. Solid supports further include thin film, membrane, polymers such as particles, beads, microspheres, microparticles, or any combination thereof.

As used herein, the term “sequencing” refers to the technique for the determination of the order of molecules, such as nucleotides or amino acids, in a ligand molecule, such as polynucleotide or polypeptide, or a sample of ligand molecules.

As used herein, the term “next generation sequencing” refers to high throughput sequencing methods that allow the sequencing of millions to billions of molecules in parallel. Examples of next generation sequencing methods include sequencing by synthesis, sequencing by ligation, sequencing by hybridization, semiconductor sequencing, and pyrosequencing. By attaching primers to a solid surface and a complementary sequence to a nucleic acid molecule, a nucleic acid molecule can be hybridized to the solid surfaces via the primer and then multiple copies can be generated in a discrete area on the solid surface by using polymerase to amplify. Consequently, during the sequencing process, a nucleotide at a particular position can be sequenced multiple times, which is referred to as depth of sequencing. Examples of high throughput nucleic acid sequencing technology include platforms provided by Illumina, MGI, Qiagen, Thermo Fisher, Genemind, and Roche.

As used herein, the term “single molecule sequencing” refers to the sequencing method wherein reads from single molecule are generated by sequencing of a single molecule of DNA. Unlike next generation sequencing methods that rely on amplification to clone many DNA molecules in parallel for sequencing in a stepwise approach, single molecule sequencing interrogates single molecules of DNA and does not require amplification. Examples of single molecule methods include single molecule real time sequencing (Pacific Biosciences), nanopore based sequencing (Oxford Nanopore), single molecule stepwise sequencing (Helicos Biosciences).

As used herein, the term “compartment” refers to a physical area or volume that separates or isolates a subset of polypeptides from a sample of polypeptides. For example, a compartment may separate an individual cell from other cells or a subset of a sample's proteome from the rest of the sample's proteome. A compartment may be aqueous compartment (e.g. droplet), a solid compartment (e.g. well or beads). The term “compartment barcode” refers to a single or double strain nucleic acid molecule of about 4 bases to about 100 bases, or any bases between, that comprises identifying information for the constitutes within one or more compartments. A compartment barcode identifies a subset of polypeptides in a sample that have been separated into the same physical compartment or group of compartments from a plurality of compartments. Thus, a compartment tag can be used. To distinguish constitutes derived from one or more compartments having the same compartment tag from those in another compartment having a different compartment tag, even after the constitutes are pooled together.

### Methods of Protein and Peptide Sequencing

Provided in some aspects are methods for protein and peptide sequencing. The methods described herein provide a highly parallelized approach for polypeptide sequencing. In some embodiments, the method described herein provide a highly multiplex approach for polypeptide sequencing.

Provided in some aspects are methods for sequencing a polypeptide, comprising the steps of: (a) providing a polypeptide immobilized on solid surfaces optionally through a sample ID DNA tag; (b) optionally binding a N-terminal amino acid (NTAA) of the polypeptide with a modification molecule; (c) contacting the polypeptide with a NTAA binding molecule with a coding DNA tag; (d) transferring the information of the coding DNA tag to a universal primer on the solid surfaces; (e) cleaving the N-terminal amino acid on the polypeptide; (f) cyclically repeating step (b) through step (e); (g) optionally amplifying the coding DNA strand into a cluster in situ on the solid surfaces; (h) decoding DNA strands on the solid surfaces.

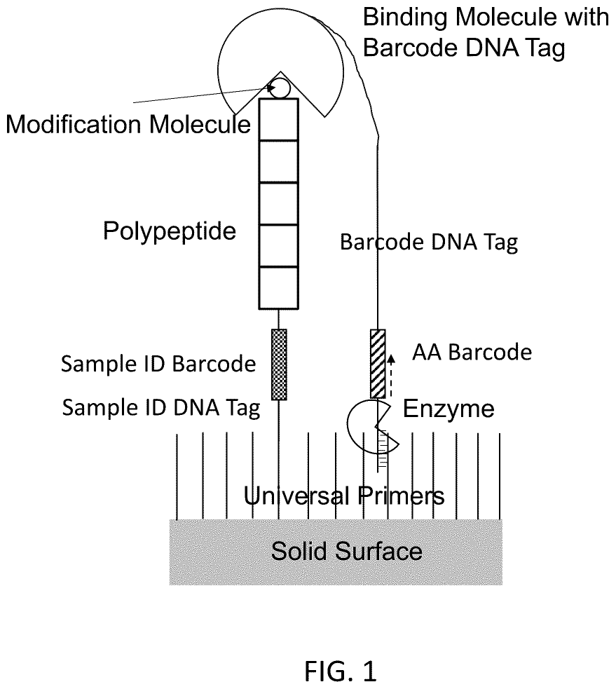

FIG. 1 illustrates a main scheme for protein and peptide sequencing in an embodiment. A polypeptide is directly immobilized on a solid surface through a sample ID DNA tag. A modification molecule is bound with a NTAA along polypeptide and then a NTAA binding molecule with a barcode DNA tag specifically binds the modified NTAA on the polypeptide. The barcode DNA tag is hybridized with a universal primer on the substrate. Enzyme extends the universal primer and transfers barcode information to the universal primer to form an extended DNA tag on the solid surface.

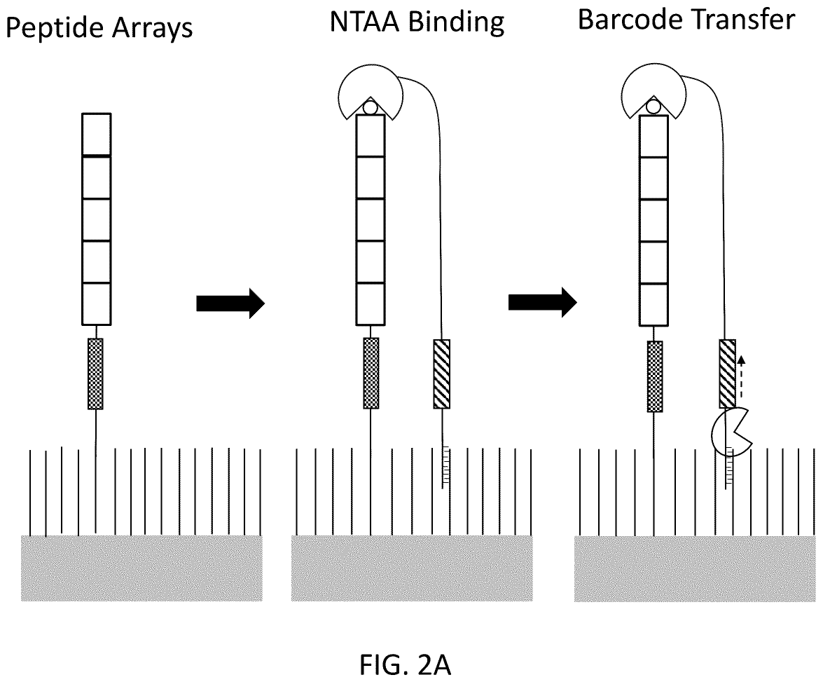

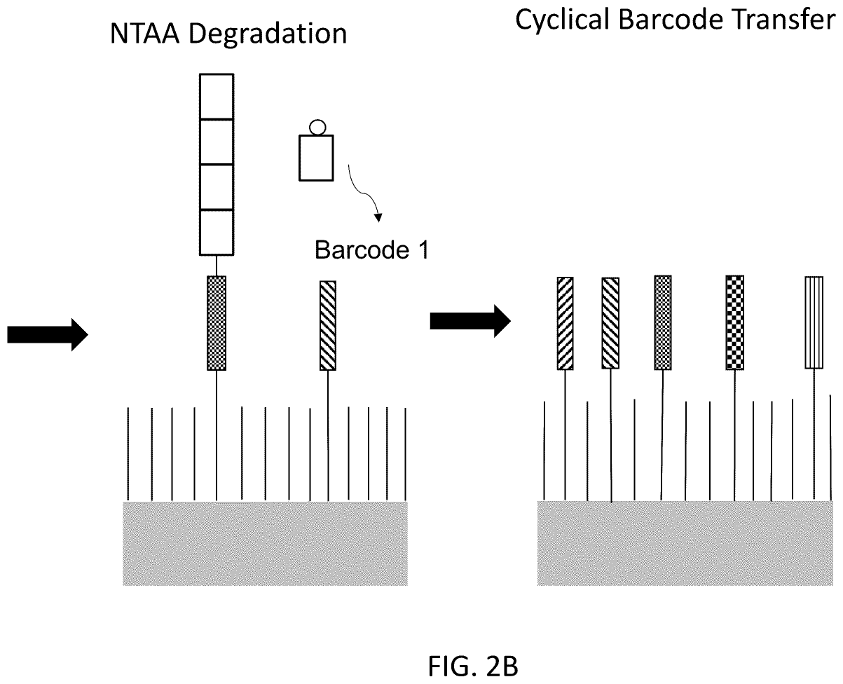

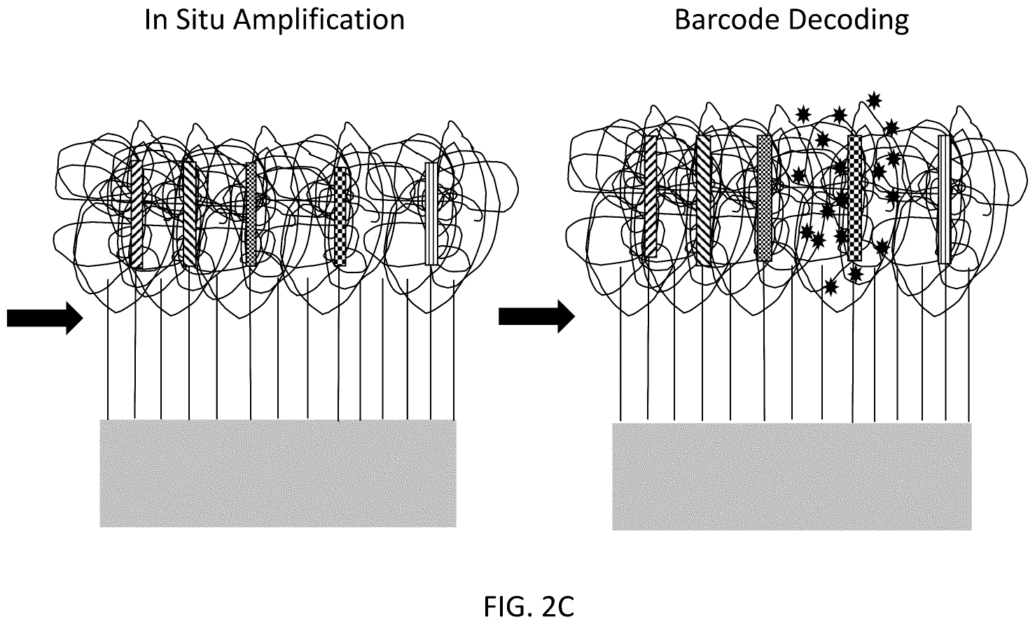

FIGS. 2A-2C illustrates the steps for protein and peptide sequencing in an embodiment. A polypeptide is immobilized on solid surface through a sample ID DNA tag. A modification molecule is bound with a NTAA along the polypeptide and then a NTAA binding molecule with a DNA coding tag specifically binds with the NTAA on the polypeptide. The DNA coding tag is hybridized with a universal primer on the substrate. Enzyme extends the universal primer and then transfer barcode information to the universal primer to form an extended DNA tag on the solid surface. The modified NTAA with binding molecule is cleaved from the polypeptide strand using a chemical method or an enzymatic method and then expose next NTAA along the polypeptide strand. The previous steps repeat cyclically to transfer all the barcodes to the solid surface. The DNA strands with barcode information are amplified into DNA clusters in situ on the solid surfaces. The amplified DNA clusters are cyclically decoded using hybridization assay or sequencing.

In some embodiments, the polypeptide is directly immobilized on the solid surface through a tethering group. A solid surface can be two-dimensional planar surface or three-dimensional surface, including, but not limited to, a bead, a microbead, an array, a glass surface, a silicon surface, a plastic surface etc. Materials for a solid surface include but are not limited to acrylamide, agarose, cellulose, glass, gold, quartz, polystryrene, polyethylene, plyethylene oxide, polysilicates, polycarbonates, Teflon, fluorocarbons, nylon, functionalized silane, collagen, polyamino acids, or any combination thereof. The tethering group includes, but is not limited to, isothiocyanate, tetrabutylammonium isothiocyanate, diphenylphosphoryl isothiocyanate, azide, alkyne, Dibenzocyclooctyne, maleimide, succinimide, thiol-thiol disulfide bond, Tetrazine, trans-cyclooctene (TCO), Vinyl, methylcyclopropene, primary amine, carboxylic acid, alkyne, acryloyl, allyl and aldehyde. The functional groups on the solid surface can covalently immobilize the polypeptide on the surfaces. The surface functional groups include, but not limited to, aldehyde, oxime, hydrazone, hydrazide, alkyne, amine, azide, acylazide, acylhalide, nitrile, nitrone, sulfhydryl, disulfide, sulfonyl halide, isothiocyanate, imidoester, N-hydroxysuccinimide ester, pentynoic acid ester.

In some embodiments, the polypeptide fragments are approximately the length from about 10 amino acids to about 70 amino acids, from about 10 amino acids to about 60 amino acids, about 10 amino acids to about 50 amino acids, about 10 amino acids to about 40 amino acids, about 10 amino acids to about 30 amino acids, about 10 amino acids to about 20 amino acids, about 20 amino acids to about 70 amino acids, about 20 amino acids to about 60 amino acids, about 20 amino acids to about 50 amino acids, about 20 amino acids to about 40 amino acids, about 20 amino acids to about 30 amino acids, about 30 amino acids to about 70 amino acids, about 30 amino acids to about 60 amino acids, about 30 amino acids to about 50 amino acids, and about 30 amino acids to about 40 amino acids.

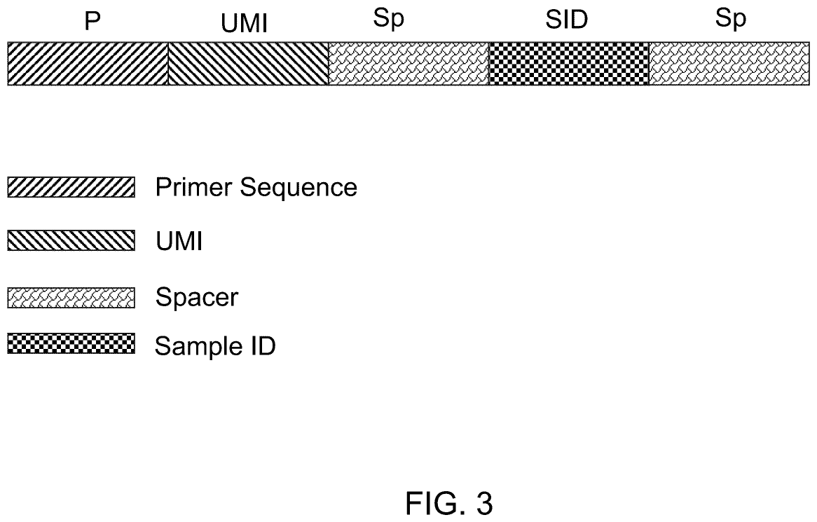

In some embodiments, the polypeptide is immobilized on the solid surfaces through a sample ID DNA tag. Provided herein is a sample ID DNA tag, comprising one or more universal primer sequences, one or more unique molecular identifier (UMI) sequences, one or more spacer sequences, one or more sample ID sequence, one or more compartment sequences, one or more sequencing cycle number sequences or any combination thereof. FIG. 3 illustrates a general overview of a sample ID DNA tag.

In some embodiments, the sample ID DNA tag is directly immobilized on the solid surface through a tethering group. The tethering group includes, but is not limited to, isothiocyanate, tetrabutylammonium isothiocyanate, diphenylphosphoryl isothiocyanate, azide, alkyne, Dibenzocyclooctyne, maleimide, succinimide, thiol-thiol disulfide bond, Tetrazine, trans-cyclooctene (TCO), Vinyl, methylcyclopropene, primary amine, carboxylic acid, alkyne, acryloyl, allyl and aldehyde. The functional groups on the solid surface can covalently immobilize the polypeptide on the surfaces. The surface functional groups include, but not limited to, aldehyde, oxime, hydrazone, hydrazide, alkyne, amine, azide, acylazide, acylhalide, nitrile, nitrone, sulfhydryl, disulfide, sulfonyl halide, isothiocyanate, imidoester, N-hydroxysuccinimide ester, pentynoic acid ester.

In some embodiments, the sample ID DNA tag is indirectly immobilized on the solid surface through a binding pair. The binding pair includes, but not limited to, an antigen and an antibody against the antigen (including its fragments, derivatives, or mimetics), a ligand and its receptor, complementary strands of nucleic acids (e.g., Poly A or Ploy T), biotin and avidin (or streptavidin or neutravidin), lectin and carbohydrates, and vice versa.

In certain embodiments, a unique molecular identifier (UMI) provides a unique identifier tag for each polypeptide to which the UMI is associated with. A UMI can be about 3 to about 40 bases, about 3 to about 30 bases, about 3 to about 20 bases, about 3 to about 10 bases, about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases or any number of bases between two aforementioned numbers of bases in length.

In some embodiments, a modification molecule binds with a N-terminal amino acid (NTAA) of a polypeptide to form a conjugate. The NTAA binding molecule is specifically binding with this conjugate. The modification molecule comprises, but not limited to, acetyl, formyl, pyroglutamic groups. In some embodiments, the binding between the modification molecule and the NTAA is covalent attachment. In some embodiments, the binding between the modification molecule and the NTAA is temporary or reversible, for example charging interaction or Val der Waals force or any combination thereof.

In some embodiments, a NTAA binding molecules directly binds with a NTAA of a polypeptide. In some embodiments, the NTAA binding molecule is a protein. The NTAA binding protein can specifically recognize amino acids of the polypeptide. The protein based NTAA binding molecule is, but not limited to, Aminoacyl-tRNA synthetase (Gamper et al. 2020), Periplasmic binding protein, Peptide transporter, Dipepetide permease, protein coupled peptide transporter, N-end rule, Clps, UBR, Transferase, engineered Aminopeptidase and antibody. In some embodiments, the NTAA binding molecule is an aptamer. It includes, but not limited to, RNA aptamer, DNA aptamer, peptide nucleic acid (PNA), and locked nucleic acid (LNA) (Jepsen et al. 2004; Siddiquee et al. 2015).

In some embodiments, any binding molecules described also comprises a coding tag containing identifying information regarding associated amino acids. A coding tag is a nucleic acid molecule of about 3 bases to about 100 bases that provides unique identifying information for its associated amino acids. A coding tag may comprise about 3 to about 90 bases, about 3 to about 80 bases, about 3 to about 70 bases, about 3 to about 60 bases, about 3 to about 50 bases, about 3 to about 40 bases, about 3 to about 30 bases, about 3 to about 20 bases, about 3 to about 10 bases, about 3 to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 50 bases, 60 bases, 70 bases, 80 bases, 90 bases, 100 bases., 200 bases, 300 bases, 400 bases, 500 bases or any number of bases between two aforementioned number of bases. A coding tag may be composed of DNA, RNA, polynucleotide analogs, or a combination thereof.

In some embodiments, each binding molecule has a unique encoder sequence with a library of binding molecules. For example, 20 unique encoder sequences may be used for a library of 20 binding molecules that identify 20 standard amino acids. Additional coding tag sequence may be used to identify modified amino acids (e.g. post translationally modified amino acids). In another example, 30 unique encoder sequences may be used for a library of 30 binding molecules that bind to the 20 standard amino acids and 10 post-translational modified amino acids. In some embodiments, two or more different binding molecules may share the same encoder sequencer.

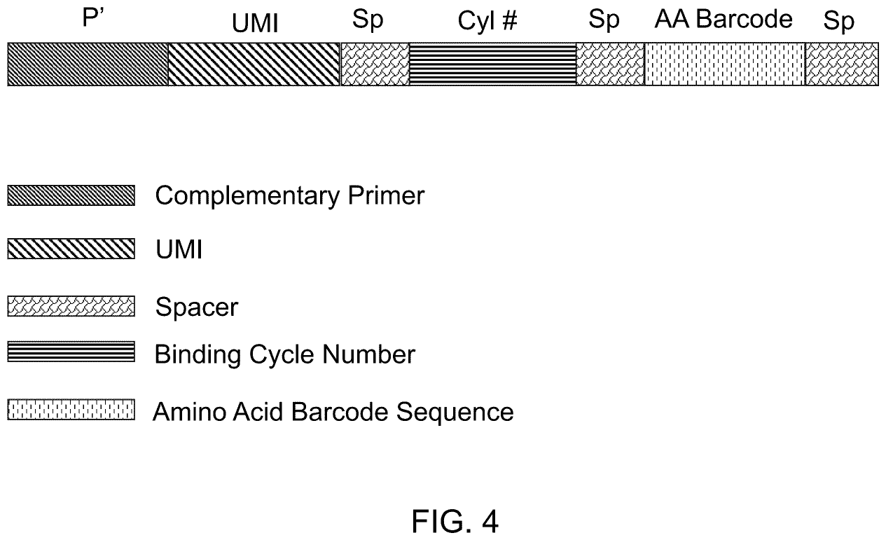

In some embodiments, the DNA tag of the binding molecules comprise one or more universal primer sequences, one or more unique molecular identifier (UMI) sequences, one or more spacer sequences, one or more sample ID sequence, one or more compartment sequences, one or more sequencing cycle number sequences or any combination thereof. FIG. 4 illustrates a general overview of a coding DNA tag of the binding molecules.

In some embodiments, the information transfer from the DNA barcode of the binding molecules to the universal primer on the solid surfaces can be accomplished using a primer extension step. A sequence on the 3″ terminus of a universal primer on the solid surfaces anneals with complementary sequence on the 3′ terminus of a DNA coding tag of the binding molecule and a polymerase extends the universal primer sequence using the annealed DNA coding tag as the template. Examples of such polymerases includes, but not limited to, Klenow, T4 DNA polymerase, T7 DNA polymerase, Bst DNA polymerase, Bca Pol, 9° N Pol and Phi 29 Pol.

In some embodiments, the information transfer from DNA barcode of the binding molecules to the universal primer on the solid surfaces can be accomplished using a ligation step. Ligation may be an enzymatic ligation reaction. Examples of ligase include, but not limited to, T3 DNA ligase, T4 DNA ligase, T7 DNA ligase, Taq DNA ligase, E. coli DNA ligase, 9° N DNA ligase, Electroligase etc. Alternatively, a ligation may be a chemical ligation reaction (Gunderson, Huang et al., 1998, El-Sgaheer, Cheong et al. 2011).

In some embodiment, an extended primer on the solid surfaces comprises an amino acid coding information within a cycle. For example, in the first binding cycle (Cycle 1), the information of the DNA barcode of the binding molecules is transferred to the universal primer 1. in the second binding cycle (Cycle 2), the information of the DNA barcode of the binding molecules is transferred to the universal primer 2 on the solid surfaces. In the nth binding cycle (Cycle N), the information of the DNA barcode of the binding molecules is transferred to the universal primer N on the solid surfaces. In some embodiment, an extended primer on the solid surfaces comprises concatenated multiple DNA barcoding information transferred from multiple cycles. In some embodiment, the information of the DNA barcode of the binding molecules is transferred to single universal primer on the solid surfaces. For example, from the first binding cycle (Cycle 1) to the Nth binding cycle (Cycle N), the information of the DNA barcode of the binding molecules is transferred to the same universal primer on the solid surfaces.

All of the extended DNA coding tags are colocalized with the associated polypeptide on the surfaces. The distance between the extended universal primers and the associated polypeptides is less than 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

In some embodiments. the universal primer on the solid surfaces comprises a universal priming site. The universal priming site is a nucleic acid sequence that may be used for priming a library amplification reaction and/or for sequencing. A universal primer may include, but is not limited to, a priming site for amplification, adaptor sequence that anneal to complementary oligonucleotides on a DNA tag of a binding molecule, a sequencing priming site, or a combination thereof. A universal primer can be about 10 bases to about 60 bases. In some embodiments, the universal primer has low melting temperature property (e.g. Poly A or Ploy T in Ma et al., PNAS, 2013, 110(35), 14320-14323). The low melting temperature may include, but is not limited to, 25° C., 30° C., 35° C., 40° C., 45° C., 50° C., 55° C., 60° C. and above, or any temperature between two aforementioned temperatures. When a binding molecule binds with a NTAA on a polypeptide at an elevated temperature above the melting temperature of the universal primers, the barcode DNA tags on the binding molecule cannot hybridize with the universal primers on the solid surfaces. After the binding molecule specifically binds with a NTAA on a polypeptide, the reaction temperature drops a room temperature or any temperature below the melting temperature of the universal primers, the barcode DNA tags on the binding molecule would hybridize with the universal primers on the solid surfaces and then DNA barcode information transfer processes start.

In some embodiments, a NTAA is cleaved by Edam degradation or enzymatic degradation. Edman chemistry is often used to cleave a NTAA on a polypeptide as illustrated in following steps.

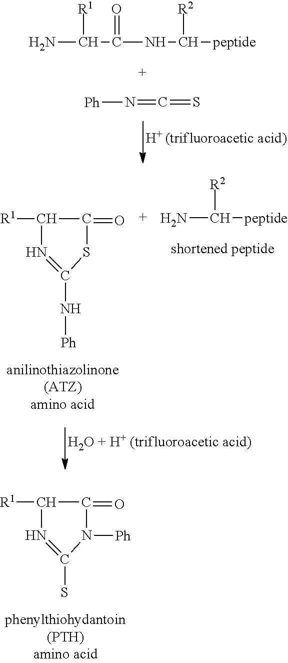

1) Coupling: Phenyl isothiocyanate (PITC) reacts with an Alpha amino group at the N-terminal end of the polypeptide chain, to form a phenylthiocarbamyl derivative of the terminal residue in basic conditions; 2) Cleavage: In the presence of strong acid, cleavage occurs at the first peptide bond, giving the peptide and the liberated first residue as the anilinothiazolinone (ATZ) form. Typically, trifluoroacetic acid (TFA) is used for the cleavage reaction. Once other reactants and products have been washed away, the shortened polypeptide can be taken through another around of coupling and cleavage to release the second residue in a cyclical fashion. The different amino acid residues, being structurally different, react at each stage with different degrees of efficiency. The overall cleavage efficiency is of the order of 95% using Edman degradation, so over the course of a number of cycles, the yield of sequences declines, and the lag gradually increases. Due to this limitation, the sequencing length of the polypeptide is usually less than 50. In some embodiments, a NTAA is cleaved using mild Edman degradation, comprising a dicholo or monochloro acid. In some embodiments, mild Edman degradation comprises triethylammonium acetate.

Another exemplary NTAA cleavage method is enzymatic degradation. Aminopeptidases are group of exopeptidases that catalyze the cleavage of the NTAA from protein or polypeptides (Ferroa, et al. 2014; Gozales and Robert-Baudouy 1996; Sanderink et al. 1988). Aminopeptidases vary in size, specificity, activity and biophysical property. Many aminopeptidases have broad specificity and degrade any NTAA, some are amino acid specific and can only catalyze the removal of a specific amino acid such as Ala, Pro, Gly, and Met (Gozales and Robert-Baudouy 1996).

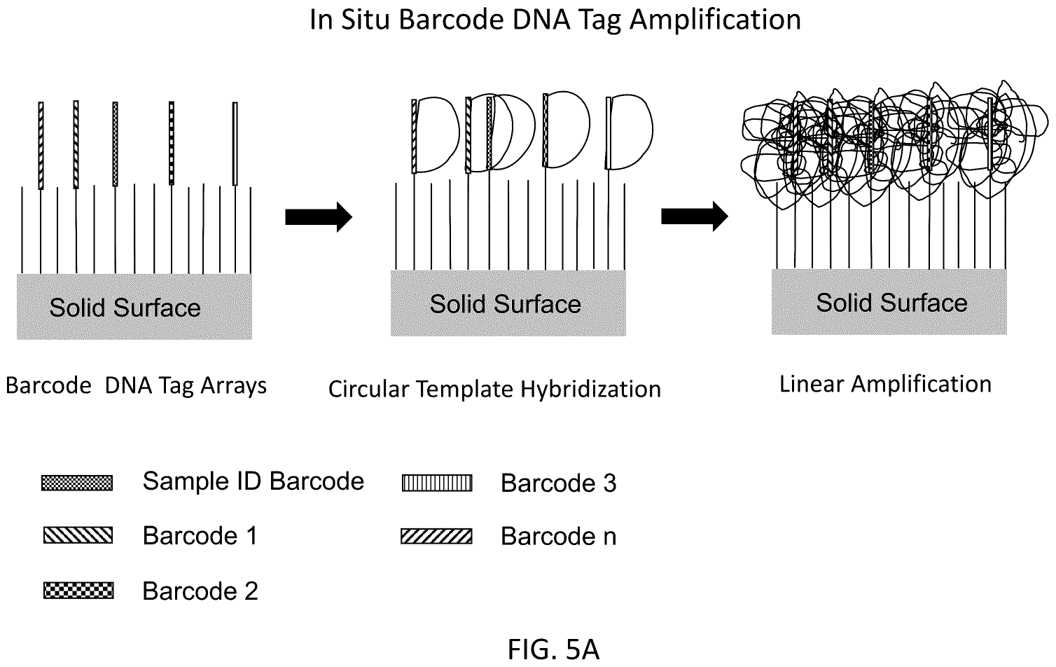

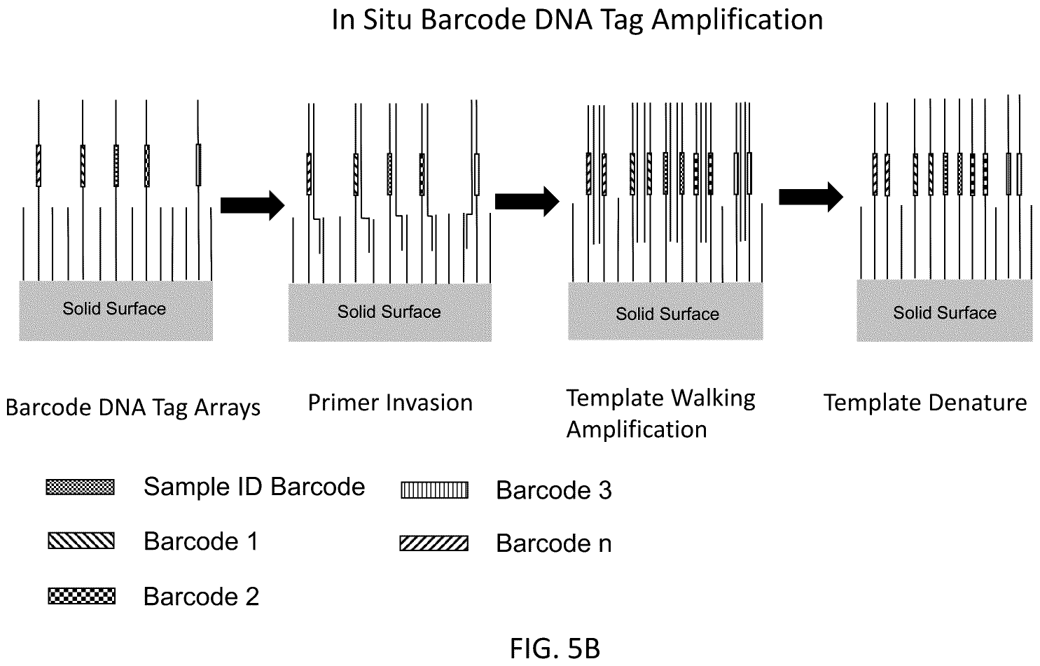

In some embodiments, exemplary DNA barcode amplification method in present disclosure may be isothermal amplification such as Rolling Cycle Amplification (RCA). RCA is an isothermal DNA amplification method that can rapidly synthesize multiple copies of circular molecules of DNA. RCA is initiated by an initiator protein encoded by the plasmid or bacteriophage DNA, which nicks one strand of the double stranded, circular DNA molecule at a site called the double strand origin. The initiator protein remains bound to the 5′ phosphate end of the nicked strand and the free 3′ hydroxyl end is released to serve as a primer for DNA synthesis by DNA polymerase. Using the unnicked strand as a template, amplification proceeds around the circular DNA molecule, displacing the nicked strand as single stranded DNA. Continued DNA amplification can produce multiple single stranded linear copies of the original circular DNA template (FIG. 5A).

Another exemplary DNA barcode amplification method is Template Walking amplification (Ma et al., PNAS, 2013, 110(35), 14320-14323). Nicked templates are captured by the surface immobilized primers. Surface primers are extended to the full length of the template by strand displacement enzyme at 37° C. Template invasion by solution phase primers and template walk to nearby surface primer to form two copies of the template. The process cycle repeats to replicate a few thousand copies of the template clusters at 60° C. and 30 mins at single spot (FIG. 5B). The detail processes are described in U.S. Pat. No. 10,233,488 B2, which is incorporated herein by reference in its entirety.

Another exemplary molecule amplification method in present disclosure may be isothermal amplification such as Recombinase Polymerase Amplification (RPA). The RPA reaction exploits enzymes known as recombinases, which form complexes with oligonucleotide primers and pair the primers with their homologous sequences in duplex DNA. A single-stranded DNA binding (SSB) protein binds to the displaced DNA strand and stabilizes the resulting D loop. DNA amplification by polymerase is then initiated from the primer, but only if the target sequence is present. Once initiated, the amplification reaction progresses rapidly, so that starting with just single copy of DNA or molecule, the highly specific DNA amplification reaches detectable levels within minutes at 37 -42° C.

Other useful methods for amplifying nucleic acids are bridge amplification, loop mediated isothermal amplification (LAMP), strand displacement amplification (SDA) and Multiple Displacement Amplification (MDA).

In some embodiments, sample ID DNA tags and extended DNA barcode tags derived from the same polypeptides are colocalized on the solid surface. The distance between all of the DNA tags, including the sample ID DNA tags and the extended DNA barcode tags, associated with the same polypeptides is less than 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances. After the amplification, these amplified clusters derived from the same polypeptides are also colocalized on the surfaces. The distance between these clusters amplified from these DNA tags, including the sample ID DNA tags and the extended DNA barcode tags, is less than 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

In some embodiments, DNA barcode is cyclically decoded by primer hybridization assay using dye labeled detection probes. There are five types of nucleic acid hybridization assay: sandwich hybridization assay, competitive hybridization assay, hybridization ligation assay, dual ligation hybridization assay (DLA) and nuclease hybridization assay. In the sandwich hybridization ELISA assay format, complementary oligonucleotide capture hybridizes with a nucleic acid analyte and a labeled detection probe hybridizes with the nucleic acid analyte to form the sandwich format for detection. The completive hybridization assay relies on complementarity, where the capture probe competes between the analyte and the tracer, a labelled oligonucleotide analog the analyte. In the hybridization ligation assay, a template probe replaces the capture probe in the sandwich assay for immobilization to the solid support. The template probe is fully complementary to the oligonucleotide analyte and is intended to serve as a substrate for T4 DNA ligase-mediated ligation. The template probe has an additional stretch complementary to a ligation probe so that the ligation probe will ligate onto the 3′ end of the analyte. The ligation probe is similar to a detection probe in that it is labelled for detection. The dual ligation hybridization assay extends the specificity of the hybridization ligation assay to a specific method for the parent compound. The DLA is intended to quantify the full-length, parent oligonucleotide compound only, with both intact 5′ and 3′ ends. A capture probe and a detection probe are ligated at the 5′ and 3′ ends of the analyte by the joint action of T4 DNA ligase and T4 polynucleotide kinase. The nuclease hybridization assay is a nuclease protection assay-based hybridization ELISA. In the nuclease hybridization assay, the oligonucleotide analyte is captured onto the solid support via a fully complementary cutting probe. After enzymatic processing by S1 nuclease, the free cutting probe and the cutting probe hybridized to metabolite, i.e, shortmers of the analyte are degraded, allowing signal to be generated only from the full-length cutting probe-analyte duplex.

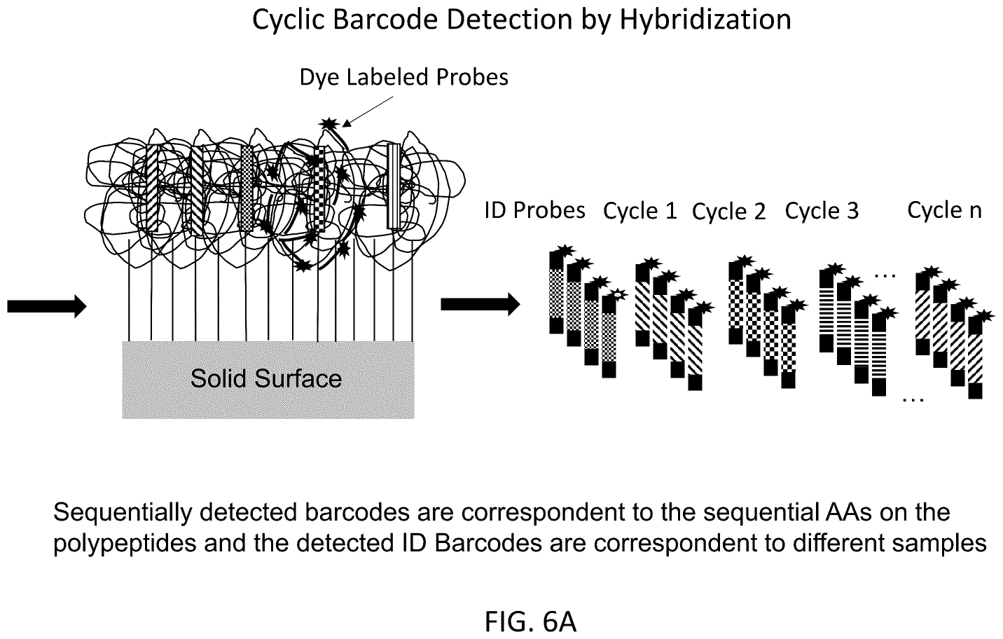

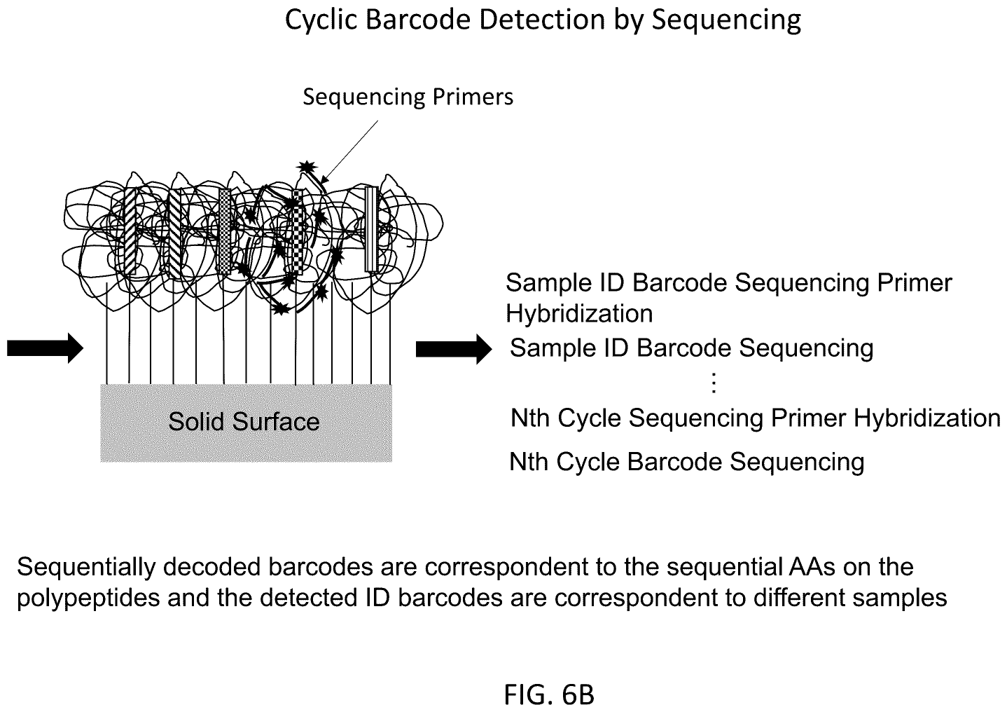

FIG. 6A illustrates cyclical barcode detection of the DNA tags on the solid surface using hybridization assay. An ID barcode probe is to decode sample information in the cluster and A barcode probe is to decode the information of the DNA coding tag in the same cluster on the solid surface. The ID barcode probes are specific for every sample and the barcode probes are specific sequence of nucleic acids for each cycle. For example, every sample in a sequencing run has been assigned a unique ID barcode. In Cycle 1, 20 amino acids have a batch of 20 specific barcode probes to detect the NTAAs that are cleaved in Cycle 1 after detection along the polypeptides. In Cycle 2, 20 amino acids have second batch of 20 specific barcode probes to detect the NTAAs that are cleaved in Cycle 2 after detection along polypeptides. In Cycle n, 20 amino acids have nth batch of 20 specific barcode probes to detect the NTAAs that are cleaved in Cycle n after detection along the polypeptides. Using these ID barcodes and different batches of barcode probes, the information of samples and the sequence of polypeptides can be decoded using hybridization assays.

In some embodiments, DNA barcode is cyclically decoded by sequencing. Both sample ID DNA tag and extended DNA coding tags on solid surface have sequencing priming sites. In some embodiments, the sample ID DNA tag and the extended DNA coding tags share the same sequencing primer. Using this same primer, the sample information of the sample ID DNA tag and the information of the extended DNA coding tags can be sequenced. The information of the sample ID DNA tag includes, but not limited to, one or more unique molecular identifier (UMI) sequences, one or more spacer sequences, one or more sample ID sequence, one or more compartment sequences and or any combination thereof. The information of the extended DNA coding tags includes, but not limited to, one or more universal primer sequences, one or more unique molecular identifier (UMI), a coding sequences associated specific amino acids, one or more spacer sequences, one or more sample ID sequence, one or more compartment sequences, one or more sequencing cycle number sequences or any combination thereof. In some embodiments, FIG. 6B illustrates that the sample ID DNA coding tag and the extended DNA coding tags in every cycle have unique sequencing primers. For example, the sample ID DNA tag has its own specific sequencing primer and the extended DNA coding tags in every cycle have the specific sequencing primer. Thus, the sample information can only be decoded by a specific primer for the sample ID DNA tag and the information of the extended DNA coding tags in Cycle N can only be decoded by a specific primer for Cycle N.

In some embodiments, DNA barcodes are cyclically decoded after DNA coding tags are amplified into clusters on solid surfaces. In some embodiments, DNA barcodes are cyclically decoded at single molecule level without cluster amplification.

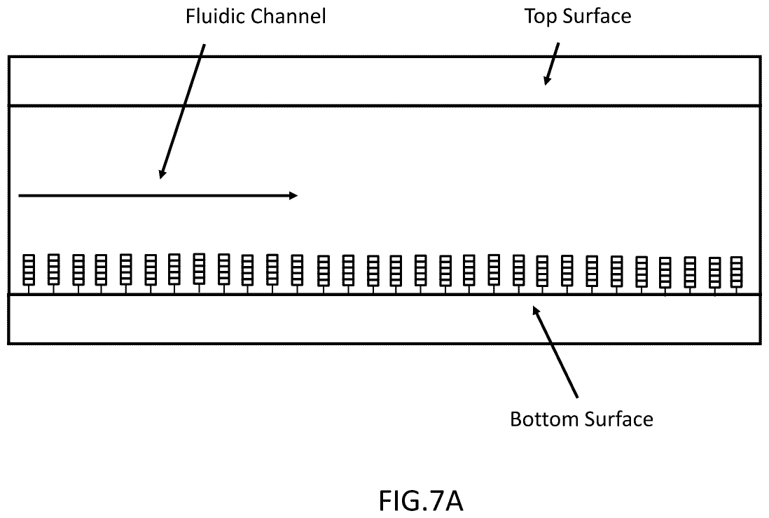

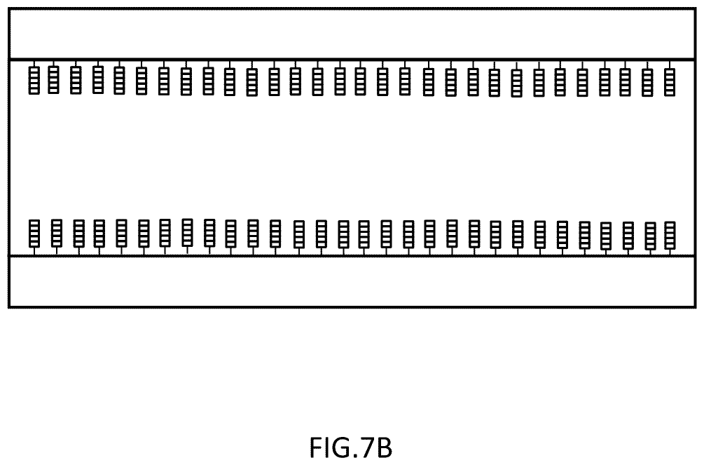

In some embodiments, polypeptides are immobilized on solid surfaces in a sequencing flow cell. FIG. 7A illustrates the typical configuration of the sequencing flow cell: a top slide, a fluidic channel and a bottom slide and an inlet and an outlet. In some embodiments, polypeptides are immobilized on the top surface of the bottom slide. In some embodiments, polypeptides are immobilized on both the bottom surface of the top slide and the top surface of the bottom slide to double the throughput (shown in FIG. 7B).

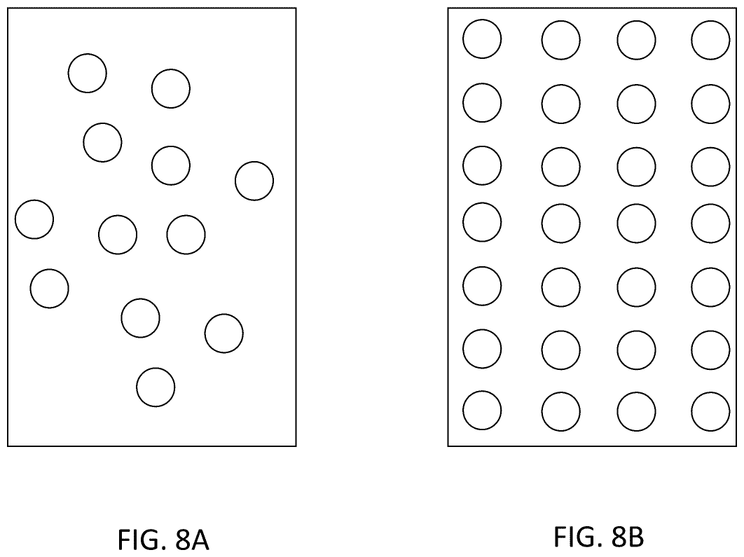

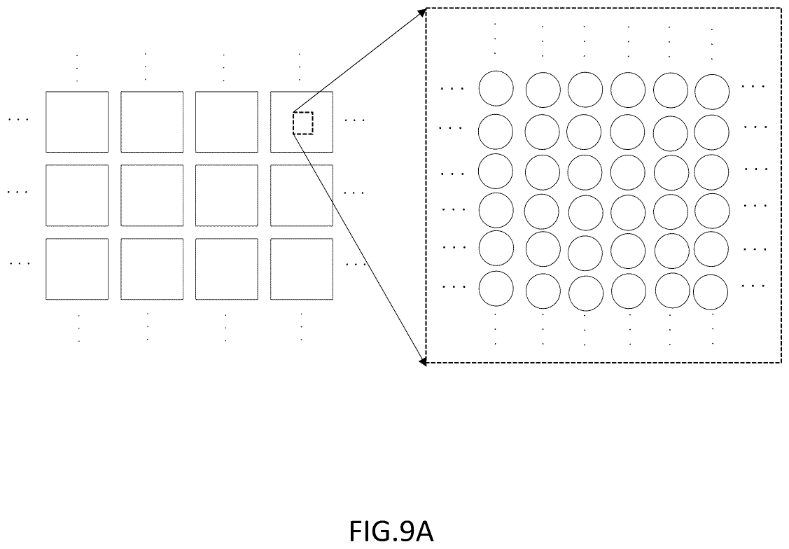

In some embodiments, polypeptides are randomly immobilized on the solid surfaces in a sequencing flow cell (shown in FIG. 8A). In certain embodiments, the polypeptides can be spaced appropriately to reduce the occurrence of or prevent a cross reaction event. The space among the polypeptides on the solid support is about 50 nm to about 500 nm, or about 50 nm to about 400 nm, or about 50 nm to about 300 nm, or about 50 nm to about 200 nm, or about 50 nm to about 100 nm. In some embodiments, multiple polypeptides are spaced apart on the surface of a solid surfaces with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm.

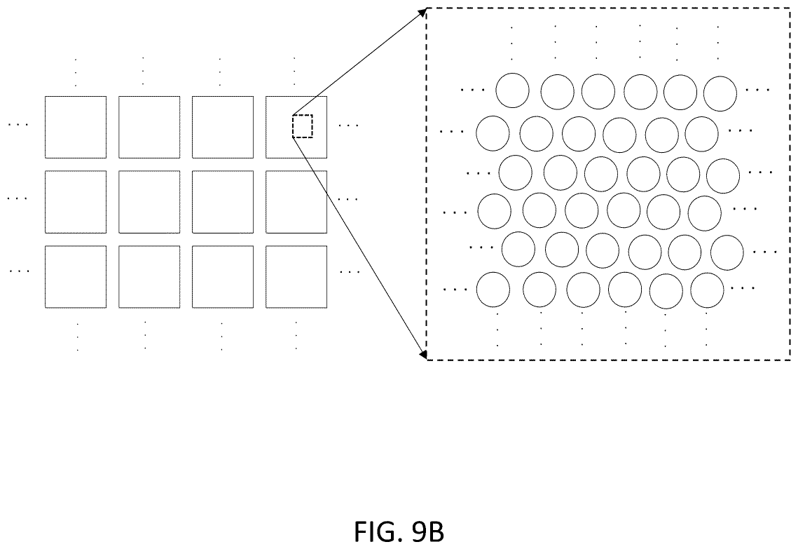

In some embodiment, polypeptides are immobilized in a regular array format on a slide in a sequencing flow cell (shown in FIG.8B). The dimension of the spots may be 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1micron and above or any dimensions between two aforementioned dimensions. The pitch of the spots may be 100 nm, 200 nm, 300 nm, 400 nm, 500 nm, 600 nm, 700 nm, 800 nm, 900 nm, 1micron and above or any dimensions between two aforementioned dimensions. The shape of the spot can be round, square, tetragon, diamond, hexagon or any shapes that are not included in this disclosure. The arrangement of the spots can be parallel (shown in FIG. 9A) or hexagonal (shown in FIG. 9B) or any formats that are not included in this disclosure. Moreover, the particular orientation of the spots, sections, lanes and so forth may differ from those illustrated in FIGS. 9A-9B. In some embodiments, the lanes and the sections are contiguous and thus not be separated by open areas.

### METHODS AND APPARATUS FOR PROTEIN AND PEPTIDE SEQUENCING

### References Cited

U.S. PATENT DOCUMENTS

| | | | |
|---|---|---|---|
| 8,268,554 | B2 | September 2012 | Schallmeriner et al |
| 9,435,810 | B2 | March 2014 | Havranek et al |
| 10,233,488 | B2 | March 2019 | Li et al |
| 10,371,634 | B2 | August 2019 | Rothberg et al |
| 10,465,235 | B2 | November 2019 | Gullberg et al |
| 11,034,995 | B2 | June 2021 | Soderberg et al |
| 11,435,358 | B2 | September 2022 | Marcotte et al |
| 11,513,126 | B2 | November 2022 | Chee et al |
| 2007/0218503 | A1 | September 2007 | Mitra et al |
| 2014/0349860 | A1 | November 2014 | Marcotte et al |
| 2015/0087526 | A1 | January 2013 | Hesselberth et al |
| 2020/0286584 | A1 | August 2019 | Patel et al |
| 2020/0209256 | A1 | December 2019 | Reed et al |
| 2020/0231956 | A1 | March 2020 | Callewaert et al |
| 2020/0217853 | A1 | July 2020 | Estandian et al |
| 2020/0219590 | A1 | July 2020 | Reed et al |
| 2020/0348307 | A1 | November 2020 | Beierle et al |
| 2020/0395099 | A1 | December 2020 | Meyer et al |
| 2021/0079557 | A1 | March 2021 | Pawlosky et al |
| 2021/0239705 | A1 | August 2021 | Mallick et al |
| 2021/0285941 | A1 | September 2021 | Luo et al |
| 2021/0405058 | A1 | December 2021 | Chee et al |
| 2021/0396762 | A1 | December 2021 | Chee et al |
| 2022/0290218 | A1 | March 2022 | Aksel et al |
| 2022/0127754 | A1 | April 2022 | Verespy III et al |
| 2022/0214353 | A1 | July 2022 | Chee et al |
| 2022/0290218 | A1 | September 2022 | Aksel et al |

FOREIGN PATENT DOCUMENTS

| | | | |
|---|---|---|---|
| WO | WO | 2010/065531A1 | December 2008 |
| WO | WO | 2010/065522A1 | December 2008 |
| WO | WO | 2013/112745A1 | January 2012 |
| WO | WO | 2021/236983A2 | May 2020 |
| WO | WO | 2022/040098A1 | August 2020 |

### OTHER PUBLICATIONS

Ma et al., “Isothermal Amplification Method for Next Generation Sequencing”, Proc. Natl. Acad. Sci., 110(35), 14320-14323 (2013).
Drmanac et al., “Human Genome Sequencing Using Unchained Base Read on Self-assembling DNA Nanoarrays”, Science, 327(5961), 78-81(2010).
Rothberg et al., “An Integrated Semiconductor Device Enabling Non-Optical Genome Sequencing”, Nature, 475, 348-352 (2011).
Arslan et al., “Sequencing by Avidity Enable High Accuracy with Low Reagent Consumption”, Nature Biotech., (2023).
Bentley et al., “Accurate whole human genome sequencing using reversible terminator chemistry”. Nature 456:53-59 (2008).
Zhao et al., “Single Molecule Sequencing of the M13 Virus Genome Without Amplification.”, PLOS one, 1-9, (2017).
Levene et al., “Zero-Mode Waveguides for Single-Molecule Analysis at High Concentrations.” Science 299, 682-686 (2003).
Tullman et al., “Engineering ClpS for Selective and Enhanced N-terminal Amino Acid Binding.” Applied Microbiology and Biotechnology, (2019).
Tullman et al., “Leveraging Nature's Biomolecular Designs in Next Generation Protein Sequencing Reagent Development.”, Applied Microbiology and Biotechnology, (2020). Niall et al., “Automated Edman Degradation: The Protein Sequenator.” Sequence Determination, 36, 942-1010, (1973).
Smith et al., “Peptide Sequencing by Edman Degradation.”, Encyclopedia of Life Sciences, 1-3, (2001).
Laursen et al., “Solid-Phase Edman Degradation: An Automatic Peptide Sequencer.”, Eur. J. Biochem. 20,89-102, (1971).
Swaminathan et al., “A Theoretical Justification for Single Molecule Peptide Sequencing.”, PLOS, 1-17, (2015).
Swaminathan et al., “Highly Parallel Single Molecule Identification of Proteins in Zeptomole Scale Mixtures.”, Nature Biotech., 36, 1076-1082, (2018).
Ginkel et al., “Single-Molecule Peptide Fingerprinting.”, PNAS, 115,3338-3343, (2018). Reed et al., “Real-time Dynamic Single Molecule Protein Sequencing on an Integrated Semiconductor Device.”, Science, 378, 186-192, (2022).
Gamper et al., “A Label Free Assay for Aminoacylation of tRNA.”, Genes, 11, 1173, 1-15, (2020).
Rodriques et al., “A Theoretical Analysis of Single Molecule Protein Sequencing Via Weak Binding Spectra.”, PLOS one, 1-23, (2019).
Jepsen et al., “Locked Nucleic acid: A Potent Nucleic Acid Analog in Therapeutic and Biotechnology.”, Oligonucleotides, 14, 130-146, (2004). Siddiquee et al., “A Review of Peptide Nucleic Acid.”, Adv Tech Biol Med, 2,1000131, (2015).
Gunderson et al., “Mutation Detection by Ligation to Complete N-mer DNA Arrays.”, Genome Res., 8, 1142-1153, (1998).
El-Sagheer et al., “Rapid Chemical Ligation of Oligonucleotides by the Diels-Alder Reaction.”, 9, 232-235, (2011).
Ferroa et al., “Intracellular Peptides: from Discovery to Function.”, EuPA Open Proteomics, 3, 143-151, (2014).
Gonzales et al., “Bacterial Aminopeptidases: Properties and Functions.”, FEMS Microbiol Rev, 18, 319-344, (1996).
Sanderink et al., “Human Aminopeptidases: A Review of the Literature.”, J Clin Chem Clin Biochem, 26, 795-807, (1988).

What is claimed is:

1. A method for sequencing protein and peptide, the method comprising the steps of:
(a) providing a polypeptide that is immobilized on solid surfaces;
(b) binding a N-terminal amino acid (NTAA) of the polypeptide with a modification molecule;
(c) contacting the polypeptide with a NTAA binding molecule with a DNA coding tag;
(d) transferring an information from the DNA coding tag of the NTAA binding molecule to a universal primer and then forming an extended DNA tag on the solid surfaces;
(e) cleaving the N-terminal amino acid on the polypeptide;
(f) cyclically repeating step (b) through step (e);
(g) decoding extended DNA strains on the solid surfaces.

2. The method of claim 1, wherein in step (a) the solid support comprises a bead, a porous bead, a glass surface, a silicon surface, a metal surface, or a plastic surface.

3. The method of claim 1, wherein in step (a) the polypeptide is immobilized on solid surfaces through a sample ID DNA tag.

4. The method of claim 3, wherein the sample ID DNA tag comprises one or more unique molecular identifier (UMI) sequences, one or more spacer sequences, one or more sample ID sequence, one or more compartment sequences and/or any combination thereof.

5. The method of claim 1, wherein in step (a) the method of the polypeptides is immobilized on the solid surfaces in a random format or a regular array format.

6. The method of claim 1, wherein in step (b) the modification molecule comprises acetyl, formyl, or pyroglutamic groups.

7. The method of claim 1, wherein in step (c) the binding molecule is a protein based NTAA binding molecule or a nucleic acid based aptamer.

8. The method of claim 1, wherein in step (c) the coding DNA tag comprises one or more universal primer sequences, one or more unique molecular identifier (UMI), a coding sequences associated specific amino acids, one or more spacer sequences, one or more sample ID sequence, one or more compartment sequences, one or more sequencing cycle number sequences or any combination thereof.

9. The method of claim 1, wherein in step (d) the transferring method is ligation or extension.

10. The method of claim 1, wherein in the step (d) the extended DNA tags and the sample ID DNA coding tags are colocalized with the associated polypeptides on the solid surfaces.

11. The method of claim 10, wherein the distance of the colocalized DNA tags, including the extended DNA tags and the sample ID DNA tag associated with the same polypeptide is 1 nm, 2 nm, 3 nm, 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

12. The method of claim 1, wherein in step (d) the universal primer comprises a priming site for amplification, adaptor sequence that anneal to complementary oligonucleotides on a DNA tag of a binding molecule, a sequencing priming site, or a combination thereof.

13. The method of claim 12, wherein the universal primer has low melting temperature property.

14. The method of claim 13, wherein the low melting temperature of the universal primer is 25° C., 30° C., 35° C., 40° C., 45° C., 50° C., 55° C., 60° C. and above, or any temperature between two aforementioned temperatures.

15. The method of claim 1, where in step (e) the cleaving of a NTAA is Edman degradation or enzymatic degradation.

16. The method of claim 1, wherein in step (g) the decoding method of the immobilized DNA clusters on the solid surfaces is primer hybridization assay or sequencing.

17. The method of claim 16, wherein the sequencing method is next generation sequencing or single molecule sequencing.

18. The method of claim 17, wherein the sequencing method is optical fluorescence based next generation sequencing or single molecule sequencing.

19. The method of claim 1 further comprising of the step amplifying immobilized DNA tags into clusters in situ on the solid surfaces between step (f) and step (g).

20. The method of claim 19, wherein the in-situ amplification reaction of the DNA tags is isothermal amplification or PCR.

21. The method of claim 20, wherein the isothermal amplification method comprises template walking, RPA, RCA, LAMP, SDA or MDA.

22. The method of claim 20, wherein PCR comprises bridge PCR.

23. The method of claim 19, wherein these amplified clusters derived from the same polypeptide are colocalized on the solid surfaces.

24. The method of claim 23, wherein the distance of the colocalized DNA clusters associated with the same polypeptide, including the extended DNA tags and the sample ID DNA coding tag, is 1 nm, 2 nm, 3 nm, 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

25. A method for sequencing protein and peptide, the method comprising the steps of:
(a) providing a polypeptide that is immobilized on solid surfaces;
(b) contacting the polypeptide with a NTAA binding molecule with a DNA coding tag;
(c) transferring an information from the DNA coding tag of the NTAA binding molecule to a universal primer and then forming an extended DNA tag on the solid surfaces;
(d) cleaving the N-terminal amino acid on the polypeptide;
(e) cyclically repeating step (b) through step (d);
(f) decoding extended DNA strains on the solid surfaces.

26. The method of claim 25, wherein in step (a) the polypeptide is immobilized on solid surfaces through a sample ID DNA tag.

27. The method of claim 25, wherein in step (a) the method of the polypeptides is immobilized on the solid surfaces in a random format or a regular array format.

28. The method of claim 25, wherein in step (c) the transferring method is ligation or extension.

29. The method of claim 25, wherein in the step (c) the extended DNA tags and the sample ID DNA coding tags are colocalized with the associated polypeptides on the solid surfaces.

30. The method of claim 29, wherein the distance of the colocalized DNA tags, including the extended DNA tags and the sample ID DNA tag associated with the same polypeptide is 1 nm, 2 nm, 3 nm, 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

31. The method of claim 25, wherein in the step (c) the universal primer has low melting temperature property.

32. The method of claim 31, wherein the low melting temperature of the universal primer is 25° C., 30° C., 35° C., 40° C., 45° C., 50° C., 55° C., 60° C. and above, or any temperature between two aforementioned temperatures.

33. The method of claim 25, wherein in step (f) the decoding method of the immobilized DNA clusters on the solid surfaces is primer hybridization assay or sequencing.

34. The method of claim 33, wherein the sequencing method is next generation sequencing or single molecule sequencing.

35. The method of claim 34, wherein the sequencing method is optical fluorescence based next generation sequencing or single molecule sequencing.

36. The method of claim 25 further comprising of the step amplifying immobilized DNA tags into clusters in situ on the solid surfaces between step (e) and step (f).

37. The method of claim 36, wherein the in-situ amplification reaction of the DNA tags is isothermal amplification or PCR.

38. The method of claim 37, wherein the isothermal amplification method comprises template walking, RPA, RCA, LAMP, SDA or MDA.

39. The method of claim 37, wherein PCR comprises bridge PCR.

40. The method of claim 36, wherein these amplified clusters derived from the same polypeptide are colocalized on the solid surfaces.

41. The method of claim 40, wherein the distance of the colocalized DNA clusters associated with the same polypeptide, including the extended DNA tags and the sample ID DNA coding tag, is 1 nm, 2 nm, 3 nm, 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.
